(19) United States
(12) Patent Application Publication
(10) Pub. No.: US 2010/0234814 A1
(43) Pub. Date: Sep. 16, 2010

(54) Method and Subcutaneous Apparatus for Facilitating the Replacement of an Implanted Catheter

(76) Inventors: Christopher H. Porter, Woodinville, WA (US); Claude A. Vidal, Santa Barbara, CA (US); Russ J. Redmond, Goleta, CA (US); Byron L. Moran, Santa Barbara, CA (US); Paul Kaluzniak, Simi Valley, CA (US); Abram D. Janis, Valencia, CA (US)

Correspondence Address: Arthur Freilich;Freilich, Hornbaker & Rosen, 20555 Devonshire Street, # 372, Chatsworth, CA, 91311-3343, US

(21) Appl. No.: 12/223,047

(22) PCT Filed: Jan. 29, 2007
(86) PCT No.: PCT/US07/02399

§ 371 Date: Jul. 21, 2008

Related U.S. Application Data
(60) Provisional application No. 60/764,675, filed on Feb. 1, 2006.

(51) Int. Cl. A61L 15/46

(52) U.S. Cl. 604/265

(57) ABSTRACT

A medical apparatus and method of use for implanting a catheter in a patient's body which catheter can be easily positioned, repositioned, and replaced. The apparatus includes an elongate sleeve intended to be subcutaneously implanted. The sleeve comprises a wall surrounding an interior elongate passageway which extends from a sleeve proximal end to a sleeve distal end. The sleeve outer peripheral surface carries a layer of porous material intended to be placed just under the patient's outer skin layer in contact with the dermis to promote tissue ingrowth for anchoring the sleeve and forming an infection resistant barrier. The sleeve passageway includes a sealing nib dimensioned to engage the outer surface of the catheter while permitting the catheter to slide relative to the sleeve. The sealing nib prevents deleterious material from migrating into the patient's body along the catheter outer surface.

### FIELD OF THE INVENTION

This invention relates generally to medical devices and more particularly to the use of percutaneously extending catheters for providing access to interior body sites, e.g., the central venous system for hemodialysis procedures.

### BACKGROUND OF THE INVENTION

In a variety of medical procedures, catheters are implanted through a patient's skin to provide long term access to interior body sites; e.g., blood vessels and organs. Unless adequate precautions are taken, infections and inflammation can readily occur at the catheter entry site. To mitigate such problems, a tissue integrating cuff is sometimes attached to the catheter and placed under the patient's skin to resist infection. Although such a cuff can reduce the likelihood of infection, its presence increases the difficulty of removing and/or repositioning an implanted catheter. More particularly, it is not uncommon for an implanted catheter to become damaged, e.g., clogged or kinked, over an extended period of use thus necessitating catheter removal and/or replacement. When this occurs, the cuff must be debrided thereby complicating and prolonging the surgical procedure.

The aforementioned application Ser. No. 10/821,383 describes the use of a tissue integrating structure on a percutaneously implanted medical device for anchoring the device and creating an infection resistant barrier around the device.

### SUMMARY OF THE INVENTION

The present invention is directed to a medical apparatus and method of use for implanting a catheter in a patient's body so as to allow the catheter to be easily positioned, repositioned, and replaced.

A catheter assembly in accordance with the present invention includes an elongate sleeve comprising a wall surrounding an interior elongate passageway. The passageway extends from a sleeve proximal end to a sleeve distal end. The sleeve is intended to be subcutaneously implanted through an incision in the patient's skin so that the sleeve proximal end resides just beneath the patient's outer skin layer. The sleeve outer peripheral surface carries a layer of porous material, e.g., a biocompatible mesh, as described in U.S. application Ser. No. 10/821,383, intended to be placed under the patient's outer skin layer in contact with the dermis layer to promote tissue ingrowth for anchoring the sleeve and forming an infection resistant barrier. The sleeve passageway includes an interior peripheral seal means for sealing against the outer surface of a catheter while permitting the catheter to slide in the passageway relative to the seal means. The seal means functions to prevent deleterious material from migrating into the patient's body along the catheter outer surface.

In accordance with a preferred embodiment, the peripheral seal means comprises a toroidal member defining a central bore and having one or more annular nibs extending into the bore for compliantly wiping against the catheter outer surface. The compliant nibs seal against the catheter outer surface for preventing deleterious material from migrating along the catheter outer surface into the patient's body while also allowing the catheter to slide through the bore for optimum positioning during installation.

In typical use, a physician will make an incision proximate to the patient's chest or abdomen. A surgical tunneler tool is then typically inserted through the incision to form a subcutaneous tunnel to an interior site through which a catheter can be inserted. In accordance with the invention, a sleeve is mounted on the catheter as previously described. The distal end of the sleeve is then inserted through the incision to place the sleeve proximal end and porous layer subcutaneously in contact with the dermis beneath the patient's outer skin surface. The catheter extends outwardly through the sleeve proximal end and percutaneously through the patient's skin at the incision site. By applying manual pressure against the subcutaneous sleeve, the physician is able to slide and/or rotate the catheter within the sleeve for optimum catheter positioning. When the catheter is properly positioned, sutures and/or tape can be used to hold the catheter in place against the patient's outer skin surface. With the sleeve thus implanted, the patient's subcutaneous tissue will, over time, grow into the porous layer to anchor the sleeve and form an infection resistant barrier. The porous layer may be coated or impregnated with constituents having antimicrobial and/or anti-inflammatory properties to promote healing, e.g., silver containing compounds or antibiotic eluting coatings and/or steroids.

In one preferred embodiment of the invention, the porous layer on the sleeve is covered prior to use by a disposable protective sheath of thin flexible material. The sheath prevents abrasion damage as the sleeve porous layer is inserted through the incision. The sheath is preferably configured with a projecting tab which allows the physician to readily peel the sheath away, e.g., along a preformed score line, as the sleeve is inserted through the incision to place the porous layer adjacent to the patient's dermis. After the sleeve and catheter have been implanted, subcutaneous tissue will gradually grow into the porous layer to form an infection resistant barrier around the sleeve to prevent fluid and/or other deleterious material from migrating into the body along the sleeve outer surface.

A catheter assembly implanted in accordance with the invention enables the physician at some later date (e.g., months) to replace the implanted catheter while leaving the sleeve in place. To do this, the physician can apply slight manual pressure against the patient's outer skin to hold the subcutaneous sleeve in place while pulling the old catheter from the sleeve proximal end outwardly through the incision. A new catheter can then be inserted through the incision and into the proximal end of the subcutaneous sleeve for sliding movement through the bore of the peripheral seal means. This procedure can be facilitated by running a guide wire through the old catheter before it is withdrawn. The replacement catheter can then be introduced over the guide wire. Once the replacement catheter is satisfactorily placed, the guide wire can be withdrawn. In order to avoid insult to the patient's incision site, the distal end of the replacement catheter is preferably protected by a thin disposable sheath which the physician peels away as he/she introduces the catheter distal end through the incision.

### BRIEF DESCRIPTION OF THE FIGURES

FIG. 1 is a schematic representation depicting a medical device in accordance with the invention for percutaneously implanting a catheter for an exemplary hemodialysis application;

FIG. 2 is an isometric view of a preferred catheter assembly in accordance with the invention;

FIG. 3 is an exploded view of the assembly of FIG. 2 showing a catheter in phantom together with a sleeve intended for subcutaneous implantation, a toroidal seal member for mounting in the sleeve, a layer of porous material for mounting around the sleeve outer surface, and a disposable protective sheath temporarily mounted around the porous layer;

FIG. 4 is a sectional view taken substantially along the plane 4-4 of FIG. 2;

FIG. 5 is a plan view of the protective sheath;

FIG. 6 is a sectional view taken substantially along the plane 6-6 of FIG. 5 particularly showing a performed score line;

FIGS. 7-9 show successive steps in an exemplary procedure for implanting and utilizing the catheter assembly in accordance with the invention;

FIGS. 10-12 show successive steps in an exemplary procedure for replacing an implanted catheter; and

FIG. 13 shows a cross-sectional view of the catheter assembly as implanted with the porous layer adjacent the patient's dermis.

### DETAILED DESCRIPTION

Various medical regimens relating, for example, to hemodialysis drug infusion, plasmapheresis, etc., use a percutaneously implanted catheter for delivering fluid to or extracting fluid from an interior body site. The present invention is directed to a method and apparatus for facilitating the implantation and utilization of a percutaneous catheter and for facilitating the positioning, repositioning, and replacement, or exchange, of the catheter.

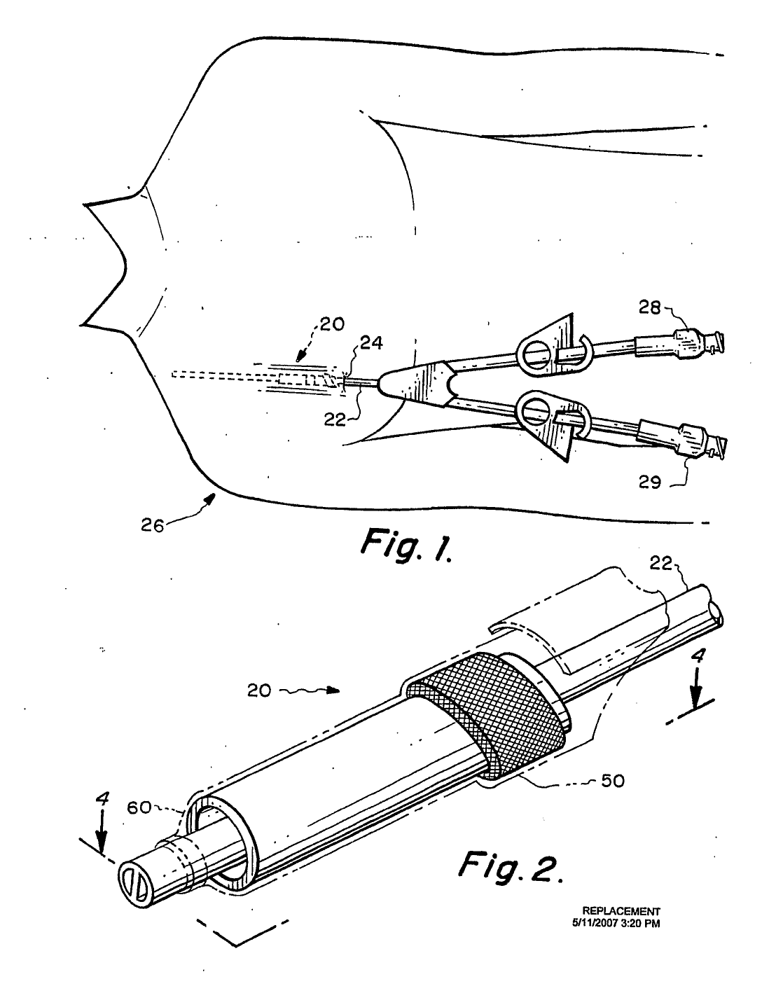

FIG. 1 schematically depicts an apparatus, or assembly, 20 in accordance with the invention which is subcutaneously implanted for allowing a catheter 22 to extend percutaneously through an incision 24 in a patient 26 undergoing an exemplary hemodialysis procedure. In such a procedure, a dual lumen catheter 22 is typically used with the two lumen respectively coupled to separate exterior flow couplers 28 and 29.

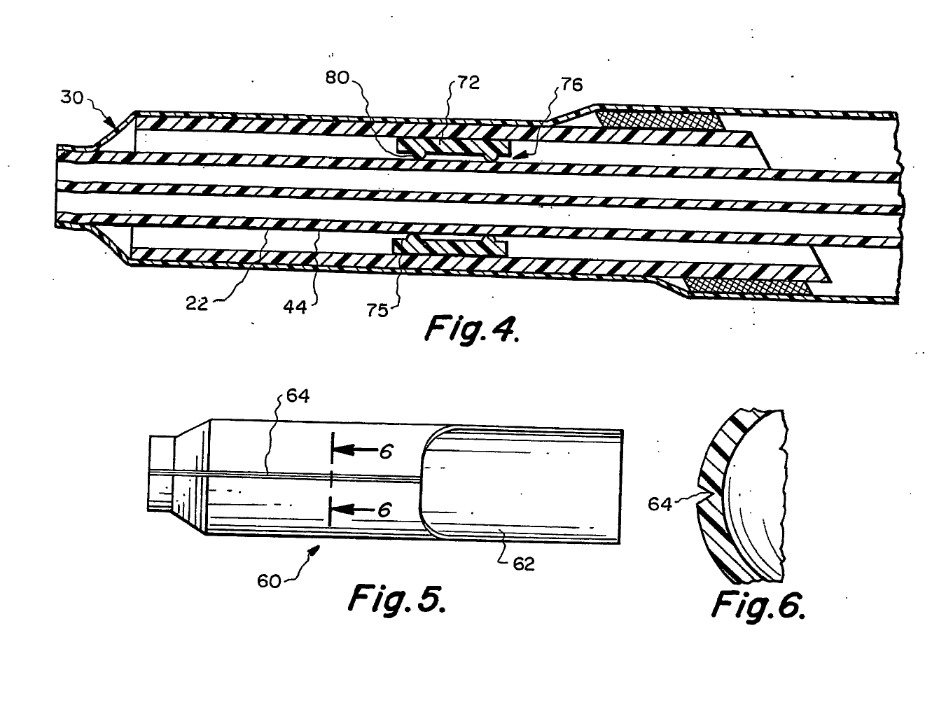

Attention is now directed to FIGS. 2-4 which depict a preferred catheter assembly 20 in accordance with the present invention. The assembly 20 is comprised of an elongate sleeve 30 formed by a sleeve wall 32 having a peripheral outer surface 34 and a peripheral inner surface 36. The inner surface 36 surrounds a passageway 38 extending from a first, or proximal, end 40 to a second, or distal, end 42. The sleeve 30 is shown mounted on a catheter 22 extending through the passageway 38. The catheter outer surface 44 and passageway wall surface 36 are closely dimensioned but the gap therebetween is sufficient to enable the catheter to slide longitudinally in the passageway 28.

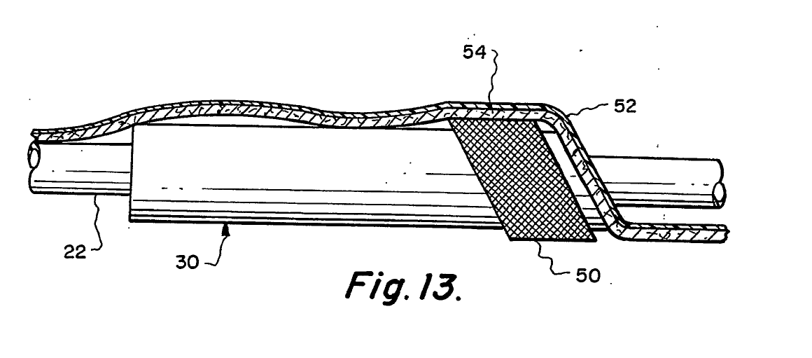

A layer 50 of porous material, e.g., titanium mesh, as described in said U.S. application Ser. No. 10/821,383, is mounted on the sleeve outer surface 34 close to the sleeve proximal end 40. In use, it is intended that the sleeve distal end 42 be inserted through the incision 24 in the patient's skin sufficiently to position the sleeve proximal end 40 and the porous layer 50 just beneath the patient's epidermis skin layer 52 and in contact with the patient's dermis layer 54 (FIG. 13). Note that the porous layer 50 is preferably oriented diagonally with respect to the axis of sleeve 30 to better conform to the patient's skin contour (FIG. 13). This orientation optimizes contact between the porous layer 50 and the patient's dermis 54 to promote, over time soft tissue ingrowth into the porous layer. This tissue ingrowth acts to form an infection resistant barrier around sleeve 30. This barrier may be enhanced by incorporating antimicrobial and/or anti-inflammatory constituents into the porous layer 50. For example, silver containing compounds and/or antibiotic eluting coatings can be used as antimicrobial agents and steroids can be used as anti-inflammatory agents.

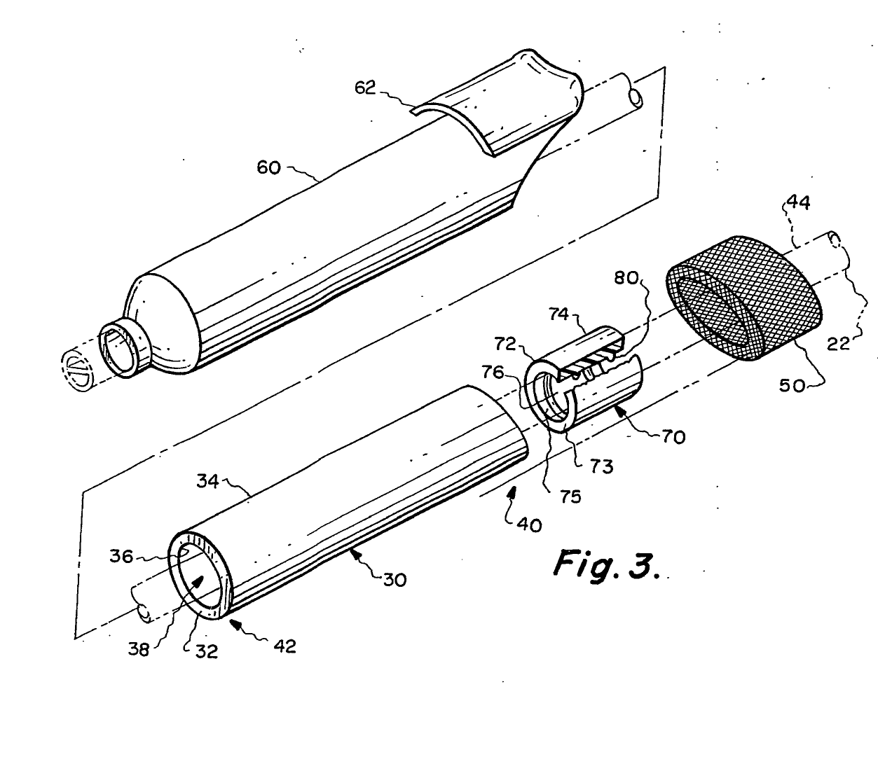

A protective sheath 60 (FIGS. 3-6) formed of thin flexible material is preferably mounted around sleeve 30 and porous layer 50 prior to use to avoid tissue abrasion damage when the sleeve distal end 42 is inserted through the patient's incision. As will be further discussed hereinafter, the sheath 60 is peeled away from the sleeve 30 by the physician as he/she inserts the sleeve through the incision. To facilitate easily peeling, the sheath 60 is preferably provided with, a pull tab 62 and a preformed score line 64 along which the sheath can readily separate.

As has previously been mentioned, in use, dermis tissue grows into the porous layer 50 to form a barrier preventing deleterious material from migrating into the patient's body along the sleeve outer surface 34. In order to prevent migration of deleterious material into the patient's body through the sleeve passageway 38 along the catheter outer surface 44, a sealing means 70 is provided proximate to the sleeve inner surface 36. A preferred sealing means 70 is comprised of a toroidal member 72, preferably formed of a polymeric material such as silicone. The toroidal member 72 is comprised of a wall 73 having an outer surface 74, and an inner surface 75 surrounding an interior bore 76 dimensioned to accommodate the catheter 22. At least one thin annular nib 80 is formed on the toroidal member inner surface 75 projecting radially into the bore 76 to bridge the gap between the sleeve inner surface 36 and the catheter outer surface 44. Each nib 80 is configured to be sufficiently axially compliant to wipe against the catheter outer surface 44 as the catheter 22 is slid through the bore 76. The nib 80 functions to prevent deleterious material from migrating along the catheter outer surface 44 into the patient's body while allowing relative sliding movement therebetween. For example only, an axial force on the catheter of 1 pound or less can be sufficient to slide the catheter relative to the nib 80.

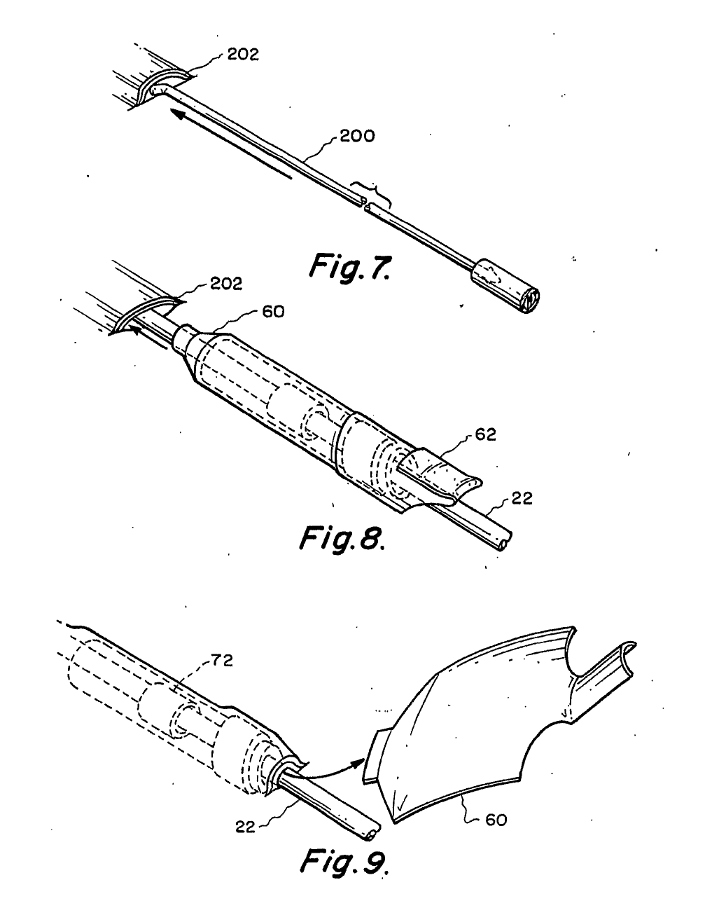

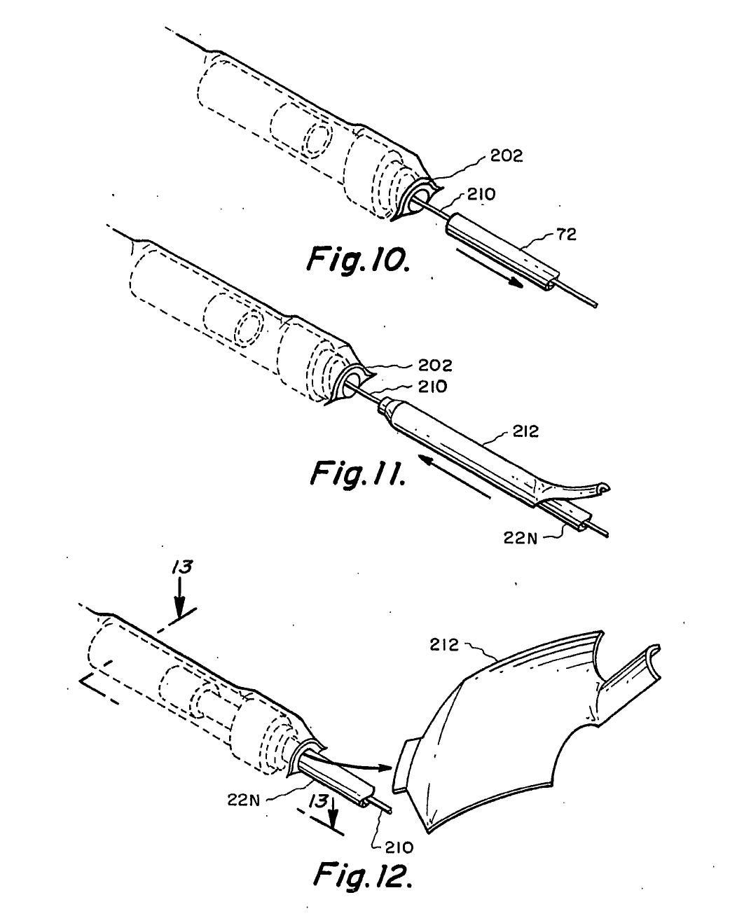

FIGS. 7-9 schematically depict successive steps in an exemplary procedure for initially implanting the catheter assembly 20 shown in FIGS. 1-6; i.e.,
FIG. 7 shows the use of a conventional tunneler tool 200 being inserted through a patient's incision 202 to form a tunnel through which the distal end of a catheter 22 is pulled by the proximal end of tool 200;
FIG. 8 shows the catheter assembly 20 with the sleeve distal end 42 and protective sheath 60 being inserted through the incision 202; and
FIG. 9 shows the catheter assembly 20 inserted further into the incision for subcutaneously positioning the sleeve proximal end 40 and porous layer 50 just beneath the patient's epidermal skin layer 52 (FIG. 13) and also shows the protective sheath 60 being peeled away (as a consequence of the physician pulling tab 62) from the sleeve 30 so that exposed the porous layer 50 is e to the patient's dermis layer 54.
FIGS. 10-12 schematically depict successive steps in an exemplary procedure for removing an old implanted catheter 22 through the sleeve 30 and replacing it with a new catheter 22N; i.e.,
FIG. 10 shows the old catheter 22 being withdrawn, along a guide wire 210 which is inserted through the catheter prior to initiating the procedure;
FIG. 11 shows the new catheter 22N being inserted along the guide wire 210 through the incision 202. Note that the distal end of the new catheter 22N preferably carries a disposable protective sheath 212, similar to aforementioned sheath 60, to minimize insult to the tissue adjacent to the incision; and
FIG. 12 shows the subcutaneous sleeve 30 with the sheath 212 being peeled away from the new catheter 22N. The guide wire 210 facilitates the movement of the catheter distal end through the sleeve passageway 38 and past the annular sealing nib 80 to its intended destination site. Once the physician has satisfactorily positioned and oriented the catheter, the guide wire 210 can be withdrawn and the exterior catheter portion can be adhered to the patient's outer skin, e.g., by tape or sutures.

FIG. 13 shows a cross-section of the subcutaneously installed catheter assembly 20 resulting from the steps represented in FIGS. 9 and 12 with the porous layer 50 contacting the patient's dermis layer 54 to promote tissue ingrowth.

From the foregoing, it should now be understood that a catheter assembly has been provided intended for subcutaneous implantation and particularly configured to facilitate the positioning, repositioning, and/or replacement of a percutaneous catheter. Although only a limited number of structural embodiments have been described, it is recognized that various modifications and alterations will occur to persons skilled in the art which fall within the spirit and intended scope of the invention as defined by the appended claims.

1. A catheter assembly configured to facilitate the positioning and/or replacement of an implanted catheter, said assembly comprising:
an elongate sleeve configured for subcutaneous implantation comprising a peripheral wall extending from a proximal end to a distal end and having an outer surface and an inner surface surrounding an elongate passageway;
an elongate catheter comprising a peripheral wall extending from a proximal end to a distal end and having an outer surface and an inner surface surrounding at least one elongate lumen;
a sealing device mounted in said sleeve passageway for engaging said catheter outer surface to prevent migration of deleterious material through said passageway while allowing said catheter to slide relative to said sealing device; and
a layer of porous material mounted on said sleeve outer surface adapted for placement in contact with a patient's dermis layer to promote tissue ingrowth and form an infection resistant barrier.

2. The assembly of claim 1 wherein said sealing device includes an annular nib extending into said sleeve passageway for engaging said catheter outer surface.

3. The assembly of claim 2 wherein said annular nib is axially compliant.

4. The assembly of claim 1 further including a protective sheath covering said porous material and configured for ready removal therefrom.

5. The assembly of claim 4 wherein said protective sheath is formed of thin flexible material; and wherein
said sheath includes a tab for pulling said sheath from said porous material.

6. The assembly of claim 4 wherein said protective sheath includes a score line.

7. The assembly of claim 1 wherein said layer of porous material incorporates antimicrobial and/or anti-inflammatory agents.

8. A method of forming a subcutaneous port for providing catheter access to interior body sites, said method comprising:
forming an incision extending through a patient's epidermis and dermis layers;
providing a sleeve having a peripheral outer surface carrying a layer of porous material and a peripheral inner surface defining a passageway;
providing a catheter having a peripheral outer surface;
sliding said catheter through said sleeve passageway;
implanting said sleeve subcutaneously to position said porous material layer adjacent said patient's dermis layer for promoting tissue ingrowth; and
sealing said passageway around said catheter to prevent the migration of deleterious material along said catheter outer surface into said body.

9. The method of claim 8 wherein said step of sealing includes mounting an annular seal in said passageway for compliantly engaging said catheter outer surface.
